(19) United States
(12) Patent Application Publication
(10) Pub. No.: US 2014/0272946 A1
(43) Pub. Date: Sep. 18, 2014

(54) Methods and Systems For DNA-Based Detection And Reporting

(71) Applicant: SRC, INC., North Syracuse, NY (US)

(72) Inventors: Adam J. Lowe, Syracuse, NY (US); Jesse W. Taylor, Baldwinsville, NY (US)

(73) Assignee: SRC, INC., North Syracuse, NY (US)

(21) Appl. No.: 13/833,538

(22) Filed: Mar. 15, 2013

(51) Int. Cl. C12Q 1/68

(52) U.S. Cl. CPC C12Q 1/6876
USPC 435/6.1; 436/164; 436/172; 435/287.2; 422/69

(57) ABSTRACT

Methods, systems, and devices for the detection and notification of chemical, biological, radiological, nuclear, and/or explosive agent. The detection and notification system uses aptamers bound to a catalyst that is activated when the aptamer binds to the agent. The activated catalyst acts on a substrate to create a signal that is detected, indicating the presence of the agent. For example, the system can be a glove worn by a user that includes a catalyst/substrate pair that, in the presence of the target agent, produces a colorimetric and/or fluorescent signal in a time-efficient manner.

### BACKGROUND

1. Field of Invention

The present invention relates to a method and system for detecting and reporting a chemical, biological, radiological, nuclear, explosive hazard, or other target of interest, and more specifically, to clothing or a device which detects and colorimetrically and/or fluorescently reports the presence of the chemical, biological, radiological, nuclear, explosive hazard, or other target of interest.

2. Description of the Related Art

There is an increasing demand for assays for detection, quantitative identification, and notification of the presence of chemical, biological, radiological, nuclear, or explosive (“CBRNE”) hazards across a broad range of disciplines, including defense, food safety, homeland security, and medical diagnostics, among many others. While there is existing technology for the detection and quantitative identification of chemical and biological hazards, these sensors are generally large, bulky, and/or slow sensor systems that require considerable time and effort to utilize or to move from one location to another. Accordingly, there is a continued need for fast, efficient, and portable sensor systems for hazard detection, as well as for systems that subsequently notify a user of any hazard that is detected.

The biological and chemical agents can be, for example, any biological agent of interest, including but not limited to those used as a biological weapon, including but not limited to numerous bacterium, virus, prion, plant disease, and fungus varieties, as well as biological toxins. Examples of chemical agents of interest include mustard gas, chloride gas, and sarin, among many other examples. Some examples of prime targets for detection by the present system include microorganisms such as Bacillus anthracis, members of the genii Burkholderia, Rickettsia, Shingella, Vibrio, and Yersinia pestis, viruses such as the smallpox virus, and toxic proteins such as ricin (from Ricinus communis) and botulinum toxin (from Clostridum botulinum), among many other possible biological or chemical agents.

There is similarly an increasing demand for rapid and efficient detection, quantitative identification, and notification of the presence of drugs and other molecular targets, including but not limited to narcotics, among others.

Aptamers are single-stranded oligonucleic acid or peptide molecules that bind to a specific target molecule. The target molecule can be, for example, a protein, nucleic acid, cell, or tissue, among many others. While some aptamers are naturally occurring, most are designed for a specific target. Due to the high affinity and specificity for their target(s) of interest, aptamers are increasingly used as diagnostic reagents. Accordingly, aptamers are a potential component of sensors for the detection and quantitative identification of chemical and biological hazards, as well as drugs and other molecular targets.

Detecting the presence of a molecular target and/or a CBRNE hazard or contaminant is only the first step in properly responding to the threat. Notifying the system or user that the hazard has been detected is an important aspect of any CBRNE system, and must function quickly and efficiently without false positives (indicating the presence of the threat to the user or system even though the threat is not present) or false negatives (failing to notify the user or system of the presence of the detected threat). According to some systems, the presence of a CBRNE hazard or contaminant may result in some sort of alarm or other notification system to be initiated, but can often be distanced from the threat or detection event in distance and/or time. In much slower systems, the presence of the CBRNE hazard or contaminant is can only be detected—and therefore reported—in a laboratory setting, thereby resulting in significant time delays in a possible response to the threat. Innovative technologies are therefore required for both the detection and reporting of the presence of CBRNE hazards and contaminants. Innovative technologies are similarly required for both the detection and reporting of the presence of drugs and other molecular targets.

### BRIEF SUMMARY

It is therefore an object and advantage to provide a method, device, and/or system for the detection and notification of the presence of a CBRNE hazard or contaminant.

It is yet another and advantage to provide a method, device, and/or system for the detection and notification of the presence of a drug or other molecular target.

It is another object and advantage to provide a method, device, and/or system for the detection and notification of the presence of a drug or other molecular target, or a CBRNE hazard or contaminant, using a wearable or portable device or article of clothing.

Other objects and advantages will in part be obvious, and in part appear hereinafter.

According to one embodiment, a method of detecting a target in a sample using an article held or worn by a user, the article comprising a target sensor, wherein the target sensor comprises a plurality of aptamers capable of binding to the target and comprising a catalyst capable of an inactive, aptamer-bound state and an active state, and further wherein the target sensor comprises a substrate configured to be acted on by the catalyst only in the active state, the method comprising the steps of: (i) contacting the article with the sample, wherein the catalyst switches from the inactive, aptamer-bound state to the active state in response to binding of the target to the plurality of aptamers, and further wherein the active state catalyst interacts with the substrate to produce a notification signal; (ii) detecting the notification signal, wherein the signal indicates the presence of the target in the sample.

According to an aspect, the notification signal is a colorimetric and/or fluorescent signal, and can, for example, result from activation of a fluorophore.

According to another aspect, the article further comprises a decontamination element, and further comprising the steps of: activating the decontamination element in response to detection of the notification signal; and decontaminating, by activation of the decontamination element, the detected target.

According to an aspect, the target is a chemical, biological, radiological, or explosive agent.

According to another aspect, the article further comprises a transmitter in communication with the target sensor, and the method comprises the step of transmitting a signal in response to detection of the target in the sample.

According to an aspect, the article comprises a plurality of different types of target sensors, where, for example, each different type of target sensor generates a unique notification signal.

According to one embodiment, a method of detecting a chemical, biological, radiological, or explosive target in a sample using an article of clothing worn by a user, the article comprising a target sensor, wherein the target sensor comprises a plurality of aptamers capable of binding to the target and comprising a catalyst capable of an inactive, aptamer-bound state and an active state, and further wherein the target sensor comprises a substrate configured to be acted on by the catalyst only in the active state, the method comprising the steps of: (i) contacting the article with the sample, wherein the catalyst switches from the inactive, aptamer-bound state to the active state in response to binding of the target to the plurality of aptamers, and further wherein the active state catalyst interacts with the substrate to produce a colorimetric and/or fluorescent notification signal; (ii) detecting the colorimetric and/or fluorescent notification signal, wherein the signal indicates the presence of the target in the sample.

According to one embodiment, a system for detecting a target in a sample using an article held or worn by a user, the system comprising: (i) a sample, the sample potentially comprising the target; and (ii) an article, the article comprising a sensor and a substrate, wherein the sensor comprises a plurality of aptamers, the plurality of aptamers capable of binding to the target and comprising a catalyst capable of an inactive, aptamer-bound state and an active state, and wherein the substrate is configured to be acted on by the catalyst only when the catalyst is in the active state; (iii) wherein the sensor is configured to generate a notification signal when the active state catalyst interacts with the substrate.

According to an aspect, the notification signal is a colorimetric and/or fluorescent signal, and can, for example, result from activation of a fluorophore, among other methods.

According to another aspect, the article further comprises a decontamination element configured to be activated in response to the notification signal.

According to an aspect, the target is a chemical, biological, radiological, or explosive agent.

According to another aspect, the system further comprises a transmitter in communication with the sensor, and the transmitter can be configured to transmit a signal in response to the notification signal.

According to an aspect, the article comprises a plurality of different types of sensors, and each different type of target sensor can generate a unique notification signal.

According to an embodiment, a system for detecting a chemical, biological, radiological, or explosive target in a sample using an article of clothing, the system comprising: (i) a sample, the sample potentially comprising the target; and (ii) an article, the article comprising a sensor and a substrate, wherein the sensor comprises a plurality of aptamers, the plurality of aptamers capable of binding to the target and comprising a catalyst capable of an inactive, aptamer-bound state and an active state, and wherein the substrate is configured to be acted on by the catalyst only when the catalyst is in the active state; (iii) wherein the sensor is configured to generate a colorimetric and/or fluorescent notification signal when the active state catalyst interacts with the substrate.

According to an aspect, the article further comprises a decontamination element configured to be activated in response to the notification signal.

According to another aspect, the system further comprises a transmitter in communication with the sensor, the transmitter configured to transmit a signal in response to the notification signal.

The details of one or more embodiments are described below and in the accompanying drawings. Other objects and advantages of the present invention will in part be obvious, and in part appear hereinafter.

### BRIEF DESCRIPTION OF THE SEVERAL VIEWS OF THE DRAWING(S)

The invention will be more fully understood and appreciated by reading the following Detailed Description in conjunction with the accompanying drawings, in which:

FIG. 1 is a schematic representation of a detection and reporting article according to an embodiment;

FIG. 2 is a schematic representation of a detection and reporting system according to an embodiment; and

FIG. 3 is a schematic flowchart of a method for detection and reporting.

### DETAILED DESCRIPTION

A detection and reporting tool, device, or article of clothing provides numerous benefits, including minimizing delays in warning times and potentially minimizing exposure to a CBRNE hazard or contaminant. These novel systems allow for significantly improved reaction time to a potential release of harmful materials thereby saving lives and decreasing total remediation time, effort, and costs. Referring now to the drawings, wherein like reference numerals refer to like parts throughout, there is seen in FIG. 1 a schematic drawing of a detection and reporting article 100 worn by a user which automatically detects and optionally decontaminates target chemical, biological, radiological, nuclear or explosive hazard(s), according to one embodiment of the method. Although detection and reporting article 100 is depicted as an item worn by a user in FIG. 1, particularly a glove, the article is not limited to an article of clothing such as a glove. For example, article 100 can be any other article of clothing, including but not limited to shirts, pants, dresses, undergarments, shoes or boots, uniforms, or hats, among many others. In addition, article 100 can be a non-clothing item such as protective gear, analytical wipes, air tanks, handheld devices such as PDAs, cellphones, or computers, among many, many other non-clothing items.

Detection Mechanism

The detection mechanism in many existing sensors involves changes in properties such as conductivity, absorbance, luminescence, fluorescence and the like. The difficulty faced by these sensors, however, include the small magnitude of the signal event which can make detection of the signal difficult or affect the selectivity or make the sensor subject to false positive readings. The detection system described herein preferably uses aptamers to sense CBRNE threats, hazards, and contaminants, as well as drugs and other small molecules. The aptamers can be, for example, protein or nucleic acid aptamers known in the art, or protein or nucleic acid aptamers created especially for this system.

For example, the aptamer can be created using any of a number of known methods in the art for isolating, identifying, or creating aptamers. While some aptamers are known to occur in nature, there are several methods used to create aptamers with high specific affinity for a target ligand such as a chemical or biological agent. The SELEX (systematic evolution of ligands by exponential enrichment) method, for example, uses multiple rounds of in vitro selection to select—and then selectively evolve—a suitable aptamer from a large library of randomly generated oligonucleotide sequences.

To enable detection, the aptamers 110 can be functionalized directly to a surface 120. According to one embodiment, aptamers 110 may be functionalized with electrically or optically conductive components such as carbon nanotubes, metal nanoparticles, or electrically conductive chemical groups such as Ferrocene or compounds containing extended conjugation. They can be attached to surface 120 using any method of functionalization known in the art, for example. Further, the aptamers can be attached by themselves, or with a stabilizing component/agent such as a sugar, trehalose, PEG, or any other stabilizer.

According to an embodiment, aptamers 110 comprise a catalytic component 170 and surface 120 comprises a substrate component 130. Catalytic component 170 and substrate component 130 may be any catalyst and substrate known in the art, however in a system the catalyst and substrate must be a reactive pair. That is, the catalyst 170 must be able to act on the substrate 130 (once the catalyst is released from the aptamer 110, as described in greater detail elsewhere herein). Among other advantages, a catalytic system provides a system in which the ratio or concentration of catalyst to substrate need not be 1:1. Accordingly, the activity of the catalyst can be exploited to increase the sensitivity or reporting capability of the overall system. Examples of catalytic reactions include, for example, enzymatic and click chemistry. According to one embodiment, the enzymatic process employs stable enzyme species in known color changing reactions to cause an indicating reaction, such as horse radish peroxidase and alkaline phosphotase and their respective substrates. According to another embodiment, the click chemistry employs a catalyst, such as a copper species, for the reaction of an azide with an aklyne. According to an embodiment, colorless azides and alkynes will react under mild conditions in the presence of the catalytic species in order to produce a colored species indicating the presence of a CBRNE or other target. Examples of other catalytic processes include, but are not limited to acid catalysis, hydroformylation, and Ziegler-Natta polymerization and hydrogenation, among others. According to one embodiment, the aptamers form a sensor and reporter system 180. The sensor and reporter can be any size depending on the requirements of the system, and can be distributed throughout the fabric or placed in a single location. For example, in FIG. 1, the glove comprises several sensor and reporter systems 180, although in other embodiments item 100 may only comprise a single, localized sensor, or the entirety of item 100 may be a sensor and reporter system 180. Indeed, there can be anywhere from one sensor to hundreds or thousands of sensors located in or on an article. In one embodiment, sensor/reporter system 180 can be placed on or embedded within a fabric such as a uniform, hat, gloves, shirt, pants, undergarment, belt, boot, shoe, or mask, among many other options. In another embodiment, sensor/reporter system 180 can be placed on or embedded within a wearable—but non-clothing—element such as a rank insignia, a warfare patch, a material patch for a swipe test, a bracelet, or a necklace, among many other options. In yet another embodiment, sensor/reporter system 180 can be placed on or embedded within an external element or tool such as a shovel, vehicle, or other device which will be used in a manner or location that could possibly come in contact with a CBRNE threat or other target. For example, sensor/reporter system 180 can be placed on the outside of a vehicle that is traveling through an area believed to be a high threat area such that it can warn individuals inside the car that the vehicle exterior has encountered a CBRNE threat or other target.

According to yet another embodiment, the system comprises a Raman spectrometer. This spectroscopic technique analyzes the molecular structure of a target by detecting the inelastic scattering of monochromatic light—typically a laser in the visible, near infrared, or near ultraviolet range—off the chemical bonds of the target. The laser light has one known wavelength, while the light scattered off the unknown species comprises multiple wavelengths, and this scattering contains information about the identity of the unknown. Indeed, since this Raman effect is specific to the chemical bonds and/or symmetry of a molecule, Raman spectroscopy provides a unique fingerprint that allows the target to be identified. A detector collects the scattered light and determines the unique fingerprint, which can then be compared to a database of fingerprints.

According to an embodiment, the system can activate the associated Raman spectrometer to conduct an analysis once the presence of a CBRNE or other target has been detected by the system (for example, once the catalyst or other molecule or component is released from the aptamer 110, or other mechanisms of activation described herein). According to another embodiment, the system can comprise a Raman enhancer that is released when the presence of a CBRNE or other target has been detected by the system. The substrate 130 can interact with the enhancer to activate, increase, or enhance detection.

Reporting Mechanism

According to one method and system, as represented by FIG. 2, sensor/reporter system 180 is exposed to a specific CBRNE or other target 140. CBRNE 140 is recognized by, and binds to, aptamer 110 to form complex 150, resulting in a conformational or other change, which thereby results in the release, relocation, or activation of catalytic component 170. Freed, relocated, or activated catalytic component 170 reacts with substrate component 130 to form complex 160 and the presence of the CBRNE is detected and reported. In the example shown in FIG. 2, the reaction of catalytic component 170 with substrate component 130 results in a colorimetric and/or fluorescent change represented by a change in the color of surface 120. According to an embodiment, rather than the entire surface 120 changing color—or any other characteristic of surface 120 changing—the reaction of catalytic component 170 with substrate component 130 can free, relocate, or activate a component or element that is capable of reporting the presence of CBRNE 140. For example, the component or element capable of reporting the presence of CBRNE could be a fluorophore. According to this embodiment, the system would require activation at the proper wavelength, as well as the ability to receive and analyze the resulting emission.

In another embodiment, the sensor/reporter system 180 can be encapsulated inside a microcapsule. For example, a plurality of microcapsules each comprising one or more sensor/reporter systems can be opened by pressure or other means just before the system is ready for use. For example, the glove depicted in FIG. 1 could be compressed or squeezed by the user to open the microcapsules and activate sensor/reporter system 180 just before the glove is to be used. According to another embodiment, sensor/reporter system 180 requires the addition of water or another component just prior to use.

In addition to or as an alternative to the colorimetric and/or fluorescent notification, sensor/reporter system 180 can be designed to provide notice to a user of the existence of a CBRNE agent or other target. For example, the system can audibly announce the CBRNE agent detected, such as triggering a speaker to say the words “ALERT, RADIATION DETECTED” when the system detects radiation, or “ALERT, PATHOGEN DETECTED” when the system detects a certain pathogen. According to one embodiment, this audible notification can be the result of detecting the colorimetric and/or fluorescent change described herein, or can be used without the colorimetric and/or fluorescent components. In addition to audible or colorimetric and/or fluorescent notification, many other forms of notification that will alert the wearer or user that a CBRNE threat, hazard, or contaminant has been detected by the system are possible.

Decontamination

The system can also be designed to release a certain decontamination measure when it detects a certain CBRNE threat or other target. For example, sensor/reporter system 180 can further comprise a storage component, such as a plurality of microcapsules, that comprise a decontamination measure agnostic to, or alternatively specific to, the detected CBRNE threat. For example, sensor/reporter system 180 can include a triggering system that releases an antiseptic agent from the decontamination microcapsules when a biological threat is detected by the system.

The decontamination microcapsules can be embedded in or on the clothing, tool, item, or external device using any known method, device, or system. The microcapsules can be formed of, for example, organic materials, such as polymeric species, or inorganic materials, such as oxides, although this list is not meant to be comprehensive. There are a wide variety of microencapsulation methods and techniques, and any one of these methods may be used to create, or encapsulate material to form, microcapsules. Further, the clothing, tool, item, or external device may comprise just one type of microcapsule, or comprise many different types of microcapsules. A system designed to decontamination multiple threats may contain many types of decontamination agents. According to one embodiment, decontamination agent is chosen from, but not limited to, one or more commercial products and/or a combination of products such as Spilfyter® Decontamination Solution 2, Supertropical bleach, EasyDecon®, QAC Decontamination Solution, M100 Sorbent Decontamination, and/or L-Gel, among many other known decontamination and/or trapping agents. According to another embodiment, decontamination agent is a commercial product, or a proprietary product or mixture. According to yet another embodiment, the decontamination agent located inside a single microcapsule is a mixture of two or more agents targeting one or more toxic compounds or materials.

As one embodiment, article 100 includes controller chip that is electrically or conductively in communication with the aptamers or a detection monitor, although other methods are possible. For example, the detection monitor system can comprise substrate components 130 functionalized to an electrically conductive surface or filament, and when catalyst 170 binds to substrate 130 to form complex 160 there can be an electrical, conductive, capacitive, or other change that is detected by the controller chip. The controller chip can be any controller, microprocessor, or other logic device that detects and processes incoming signals. For example, the controller chip can be a microprocessor programmed to initiate a response—such as a decontamination process or a notification—upon detection of a target hazard by an aptamer in the sensor. Further, as another example of a notification, once the system detects a CBRNE threat, hazard, or contaminant the system can communicate the detection to a remote location, such as a command or control center, preferably directed by the controller chip. This can be accomplished in a wired or wireless fashion using any method known in the art. As one example, the system can communicate to a local receiver located in another portion of the article 100, or located immediately nearby such as in a smartphone, computer, or other detection device.

### Example 1

### Detecting and Reporting a CBRNE Threat

According to one embodiment is a method for detection and reporting of a target hazard, such as a chemical, biological, radiological, nuclear, or explosive hazard. At step 300, the user wears, holds, or otherwise employs article 100 in a location where detection of a hazard is desirable. As just one example, step 300 could comprise a haz-mat worker wearing a detection/reporting glove, protective suit, or other article of clothing in a location where a hazard is suspected or possible. Many other articles are possible, as described above. As an alternative example, step 300 could comprise a user with detection/reporting gloves or hat in a location where a hazard is suspected or possible. Article 100 employed in the method is previously functionalized and prepared according to methods described above, and can comprise, for example, a controller chip as well as a wireless transceiver or transmitter for notification purposes. Article 100 used in method 300 can comprise a detection sensor for target hazards. For example, the sensor can be any size depending on the requirements of the system, and can be distributed throughout the fabric or placed in a single location of the glove. Further, there can be anywhere from one sensor to hundreds or thousands of sensors located in or on the glove.

At step 310 of the method in FIG. 3, one or more of the sensors functionalized to the item—such as the aptamer sensor described above—detect a target hazard such as target CBRNE 140. According to one embodiment, CBRNE 140 is recognized by and binds to aptamer 110 to form complex 150, resulting in a conformational or other change, which thereby results in the release, relocation, or activation of catalytic component 170. Freed, relocated, or activated catalytic component 170 reacts with substrate component 130 to form complex 160 and the presence of the CBRNE is detected and reported. According to another embodiment, the binding of the target CBRNE results in a specific electrical change that, in turn, is used to trigger a specific notification and/or decontamination.

At step 320, the reaction of catalytic component 170 with substrate component 130 causes a detectable colorimetric and/or fluorescent change represented by a change in the color of surface 120. According to one embodiment, the colorimetric and/or fluorescent change is only initiated, or is only detected, if a detection threshold is satisfied or surmounted. According to another embodiment, the reaction of the catalytic component with the substrate can free, relocate, or activate another component or element that is capable of reporting the presence of the CBRNE or other target. For example, the component or element capable of reporting the presence of the CBRNE could be a fluorophore that is activated—such as by separation from a quencher—by the catalyst/substrate reaction (either directly or indirectly).

According to another embodiment, the controller chip can send a signal to a transmitter, also integrated into article 100, inducing the transmitter to send a signal to a receiver that a detection event has occurred. Other information, including the specific threat detected, or quantitative information, can be included in the wireless signal. Additionally and/or alternatively, the system can be designed to specifically alert the user at/before/after step 320 of the method. As one example, the system can be designed to send a blue signal when a chemical agent is detected. As another example, the system can be set up to audibly announce the CBRNE agent detected, such as triggering a speaker to say the words “ALERT, RADIATION DETECTED” when the system detects radiation.

At optional step 330 of the method, the system triggers a decontamination process in response to detection of a target CBRNE threat or other target. For example, once the threat is detected and reported using the detection mechanisms and reporting mechanisms described above, the controller means, such as a controller chip or other logic device or microprocessor, activates a secondary system to activate microcapsules containing decontaminating agents. In one embodiment, decontamination microcapsules are embedded in or on the shirt worn by the haz-mat worker in this particular example. These microcapsules can be formed of, for example, organic materials, such as polymeric species, or inorganic materials, such as oxides, although this list is not meant to be comprehensive. There are a wide variety of microencapsulation methods and techniques, and any one of these methods may be used to create, or encapsulate material to form, microcapsules. Further, the clothing, tool, item, or external device may comprise just one type of microcapsule, or comprise many different types of microcapsules. A system designed to decontaminate multiple threats may contain many types of decontamination agents. In addition to microcapsule technology, several other types of decontamination, containment, and filtration are known in the art, and can be incorporated into the systems, methods, and devices described herein in part or in whole, including in many different combinations depending on the design of the system and/or the anticipated threats to be encountered.

Although the present invention has been described in connection with a preferred embodiment, it should be understood that modifications, alterations, and additions can be made to the invention without departing from the scope of the invention as defined by the claims.

What is claimed is:

1. A method of detecting a target in a sample using an article held or worn by a user, the article comprising a target sensor, wherein the target sensor comprises a plurality of aptamers capable of binding to the target and comprising a catalyst capable of an inactive, aptamer-bound state and an active state, and further wherein the target sensor comprises a substrate configured to be acted on by the catalyst only in said active state, the method comprising the steps of:
contacting the article with the sample, wherein the catalyst switches from the inactive, aptamer-bound state to the active state in response to binding of the target to the plurality of aptamers, and further wherein the active state catalyst interacts with the substrate to produce a notification signal;
detecting said notification signal, wherein said signal indicates the presence of said target in said sample.

2. The method of claim 1, wherein said notification signal is a colorimetric or fluorescent signal.

3. The method of claim 1, wherein said notification signal results from activation of a fluorophore.

4. The method of claim 1, wherein said article further comprises a decontamination element, and further comprising the steps of:
activating said decontamination element in response to detection of said notification signal; and
decontaminating, by activation of said decontamination element, said detected target.

5. The method of claim 1, wherein said target is a chemical, biological, radiological, or explosive agent.

6. The method of claim 1, wherein said article further comprises a transmitter in communication with said target sensor.

7. The method of claim 6, further comprising the step of:
transmitting a signal in response to detection of said target in said sample.

8. The method of claim 1, wherein said article is an article of clothing.

9. The method of claim 1, wherein said article is a handheld device.

10. The method of claim 1, wherein said article comprises a plurality of different types of target sensors.

11. The method of claim 10, wherein each different type of target sensor generates a unique notification signal.

12. The method of claim 1, further comprising the step of:
analyzing the detected target by Raman spectroscopy.

13. The method of claim 12, further comprising the step of:
releasing a Raman enhancer in response to binding of the target to the plurality of aptamers.

14. A method of detecting a chemical, biological, radiological, or explosive target in a sample using an article of clothing worn by a user, the article comprising a target sensor, wherein the target sensor comprises a plurality of aptamers capable of binding to the target and comprising a catalyst capable of an inactive, aptamer-bound state and an active state, and further wherein the target sensor comprises a substrate configured to be acted on by the catalyst only in said active state, the method comprising the steps of:
contacting the article with the sample, wherein the catalyst switches from the inactive, aptamer-bound state to the active state in response to binding of the target to the plurality of aptamers, and further wherein the active state catalyst interacts with the substrate to produce a colorimetric or fluorescent notification signal;
detecting said colorimetric or fluorescent notification signal, wherein said signal indicates the presence of said target in said sample.

15. A system for detecting a target in a sample using an article held or worn by a user, the system comprising:
a sample, said sample potentially comprising said target; and
an article, the article comprising a sensor and a substrate, wherein the sensor comprises a plurality of aptamers, said plurality of aptamers capable of binding to the target and comprising a catalyst capable of an inactive, aptamer-bound state and an active state, and wherein the substrate is configured to be acted on by the catalyst only when said catalyst is in said active state;
wherein said sensor is configured to generate a notification signal when said active state catalyst interacts with the substrate.

16. The system of claim 15, wherein said notification signal is a colorimetric or fluorescent signal.

17. The system of claim 15, wherein said notification signal results from activation of a fluorophore.

18. The system of claim 15, wherein said article further comprises a decontamination element configured to be activated in response to said notification signal.

19. The system of claim 15, wherein said target is a chemical, biological, radiological, or explosive agent.

20. The system of claim 15, wherein said system further comprises a transmitter in communication with said sensor.

21. The system of claim 20, wherein said transmitter is configured to transmit a signal in response to said notification signal.

22. The system of claim 15, wherein said article is an article of clothing.

23. The system of claim 15, wherein said article is a handheld device.

24. The system of claim 15, wherein said article comprises a plurality of different types of sensors.

25. The system of claim 24, wherein each different type of target sensor generates a unique notification signal.

26. The system of claim 15, further comprising:
a Raman spectrometer.

27. The system of claim 26, further comprising:
a Raman enhancer, wherein said Raman enhancer enhances Raman spectroscopy in response to binding of the target to the plurality of aptamers.

28. A system for detecting a chemical, biological, radiological, or explosive target in a sample using an article of clothing, the system comprising:
a sample, said sample potentially comprising said target; and
an article, the article comprising a sensor and a substrate, wherein the sensor comprises a plurality of aptamers, said plurality of aptamers capable of binding to the target and comprising a catalyst capable of an inactive, aptamer-bound state and an active state, and wherein the substrate is configured to be acted on by the catalyst only when said catalyst is in said active state;
wherein said sensor is configured to generate a colorimetric or fluorescent notification signal when said active state catalyst interacts with the substrate.

29. The system of claim 28, wherein said article further comprises a decontamination element configured to be activated in response to said notification signal.

30. The system of claim 28, wherein said system further comprises a transmitter in communication with said sensor, said transmitter configured to transmit a signal in response to said notification signal.
